SEQUENCE LISTING (1 sequence)

SEQ ID NO: 1 - DNA, 25 bp, synthetic construct
  misc_feature 1..25: note = Synthetic DNA
  source 1..25: mol_type = other DNA
ctaaccctaaccctaaccctaaccc

(19) United States
(12) Patent Application Publication
(10) Pub. No.: US 2024/0021272 A1
(43) Pub. Date: Jan. 18, 2024

(54) SYSTEMS AND METHODS FOR IDENTIFYING SEQUENCE VARIATION

(71) Applicant: LIFE TECHNOLOGIES CORPORATION, Carlsbad, CA (US)

(72) Inventors: Fiona HYLAND, San Mateo, CA (US); Eric TSUNG, Needham, MA (US); Vasisht TADIGOTLA, Brookline, MA (US); Zheng ZHANG, Arcadia, CA (US); Dumitru BRINZA, Montara, CA (US); Onur SAKARYA, Redwood City, CA (US); Xing XU, Chicago, IL (US)

(21) Appl. No.: 18/330,737

(22) Filed: Jun. 7, 2023

Related U.S. Application Data
(63) Continuation of application No. 16/530,015, filed on Aug. 2, 2019, now abandoned.
Application No. 16/530,015 is a continuation of application No. 13/623,709, filed on Sep. 20, 2012, now abandoned.
(60) Provisional application No. 61/644,771, filed on May 9, 2012.
(60) Provisional application No. 61/584,391, filed on Jan. 9, 2012.
(60) Provisional application No. 61/545,450, filed on Oct. 10, 2011.
(60) Provisional application No. 61/536,967, filed on Sep. 20, 2011.

(51) Int. Cl. G16B 30/10; G16B 30/00

(52) U.S. Cl. CPC G16B 30/10; G16B 30/00

(57) ABSTRACT

Systems and method for determining variants can receive mapped reads, and call variants. In embodiments, flow space information for the reads can be aligned to a flow space representation of a corresponding portion of the reference. Reads spanning a position with a potential variant can be grouped and a score can be calculated for the variant. Based on the scores, a list of probable variants can be provided. In various embodiments, low frequency variants can be identified where multiple potential variants are present at a position.

### RELATED APPLICATIONS

This application is a continuation of U.S. application Ser. No. 16/530,015 filed Aug. 2, 2019, which is a continuation of U.S. application Ser. No. 13/623,709 filed Sep. 20, 2012 (now abandoned), and claims priority to U.S. application No. 61/644,771 filed May 9, 2012, U.S. application No. 61/584,391 filed Jan. 9, 2012, U.S. application No. 61/545,450 filed Oct. 10, 2011, and U.S. application No. 61/536,967 filed Sep. 20, 2011, which disclosures are herein incorporated by reference in their entirety.

### SEQUENCE LISTING

This application hereby incorporates by reference the material of the electronic Sequence Listing filed concurrently herewith. The material in the electronic Sequence Listing is submitted as an XML file entitled “TP107519USCON2_SL.xml” created on Sep. 1, 2023, which has a file size of 2,320 bytes, and is herein incorporated by reference in its entirety.

### FIELD

The present disclosure generally relates to the field of nucleic acid sequencing including systems and methods for identifying genomic variants using nucleic acid sequencing data.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible. These goals can be reached through the use of sequencing platforms and methods that provide sample preparation for samples of significant complexity, sequencing larger numbers of samples in parallel (for example through use of barcodes and multiplex analysis), and/or processing high volumes of information efficiently and completing the analysis in a timely manner. Various methods, such as, for example, sequencing by synthesis, sequencing by hybridization, and sequencing by ligation are evolving to meet these challenges.

Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

Exemplary applications of NGS technologies include, but are not limited to: genomic variant detection, such as insertions/deletions, copy number variations, single nucleotide polymorphisms, etc., genomic resequencing, gene expression analysis and genomic profiling.

Of particular interest are improved systems and methods for detecting low frequency genomic variants, such as variants that have a frequency in the sample of less than about 50%). Recent advances in genotyping technologies have resulted in a better understanding of common human sequence variation, which has led to the identification of many novel genetic determinants of complex traits/diseases. Nevertheless, despite these successes, much of the genetic component of these traits/diseases remains incomplete. Although there may be many undiscovered polymorphisms associated with complex traits/diseases, the “common-disease common-variant” paradigm may not provide a complete picture. Rare (low frequency) variants may also provide a significant causal genetic component.

From the foregoing it will be appreciated that a need exists for systems and methods that can detect low frequency genomic variants using nucleic acid sequencing data.

### DRAWINGS

For a more complete understanding of the principles disclosed herein, and the advantages thereof, reference is now made to the following descriptions taken in conjunction with the accompanying drawings, in which:

FIG. 1 is a block diagram that illustrates an exemplary computer system, in accordance with various embodiments.

FIG. 2 is a schematic diagram of an exemplary system for reconstructing a nucleic acid sequence, in accordance with various embodiments.

FIG. 3 is a flow diagram illustrating an exemplary method of calling variants, in accordance with various embodiments.

FIG. 4 is a schematic diagram of an exemplary variant calling system, in accordance with various embodiments.

FIG. 5 is a flow diagram illustrating an exemplary method of realigning a read and a target sequence in flow space, in accordance with various embodiments.

FIG. 6 is a flow diagram illustrating an exemplary method of trimming primer regions from reads, in accordance with various embodiments.

FIGS. 7A and 7B are exemplary flowcharts showing a method for detecting low frequency variants in nucleic acid sequence reads, in accordance with various embodiments.

FIG. 8 provides exemplary flow space, base space, and color space representations for a nucleic acid sequence, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for detecting variants are described herein.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

It will be appreciated that there is an implied “about” prior to the temperatures, concentrations, times, number of bases, coverage, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, “a” or “an” also may refer to “at least one” or “one or more.”

A “system” sets forth a set of components, real or abstract, comprising a whole where each component interacts with or is related to at least one other component within the whole.

A “biomolecule” may refer to any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the Personal Genome Machine (PGM) and SOLiD Sequencing System of Life Technologies Corp. provide massively parallel sequencing with enhanced accuracy. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference. The PGM System and associated workflows, protocols, chemistries, etc. are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phrase “ligation cycle” refers to a step in a sequence-by-ligation process where a probe sequence is ligated to a primer or another probe sequence.

The phrase “color call” refers to an observed dye color resulting from the detection of a probe sequence after a ligation cycle of a sequencing run.

The phrase “color space” refers to a nucleic acid sequence data schema where nucleic acid sequence information is represented by a set of colors (e.g., color calls, color signals, etc.) each carrying details about the identity and/or positional sequence of bases that comprise the nucleic acid sequence. For example, the nucleic acid sequence “ATCGA” can be represented in color space by various combinations of colors that are measured as the nucleic acid sequence is interrogated using optical detection-based (e.g., dye-based, etc.) sequencing techniques such as those employed by the SOLiD System. That is, in various embodiments, the SOLiD System can employ a schema that represents a nucleic acid fragment sequence as an initial base followed by a sequence of overlapping dimers (adjacent pairs of bases). The system can encode each dimer with one of four colors using a coding scheme that results in a sequence of color calls that represent a nucleotide sequence.

The phrase “flow space” refers to a representation of the incorporation event or non-incorporation event for a particular nucleotide flow. For example, flow space can be a series of zeros and ones representing a nucleotide incorporation event (a one, “1”) or a non-incorporation event (a zero, “0”) for that particular nucleotide flow. For incorporation events where a number of consecutive nucleotides is greater than one, the flow space representation can include an integer greater than one corresponding to the number of consecutive nucleotides incorporated. It should be understood that zeros and ones are convenient representations of a non-incorporation event and a nucleotide incorporation event; however, any other symbol or designation could be used alternatively to represent and/or identify these events and non-events.

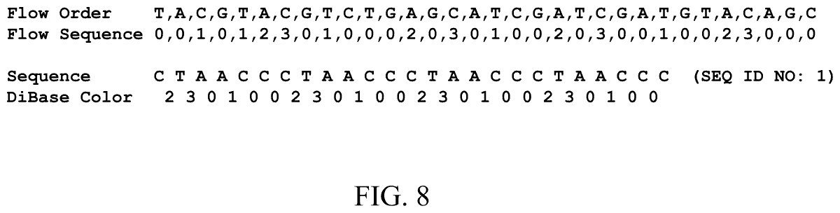

The phrase “base space” refers to a representation of the sequence of nucleotides. The term “altspace” refers to a non-base space representation, such as color space or flow space. FIG. 8 provides a flow space representation (including the nucleotide flow), a base space representation, and a color space representation for the nucleic acid sequence CTAACCCTAACCCTAACCCTAACCC (SEQ ID NO: 1).

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The techniques of “paired-end,” “pairwise,” “paired tag,” or “mate pair” sequencing are generally known in the art of molecular biology (Siegel A. F. et al., Genomics. 2000, 68: 237-246; Roach J. C. et al., Genomics. 1995, 26: 345-353). These sequencing techniques provide for the determination of multiple “reads” of sequence information from different regions on a polynucleotide strand. Typically, the distance, such as an insert region or a gap, between the reads or other information regarding a relationship between the reads is known or can be approximated. In some situations, these sequencing techniques provide more information than does sequencing stretches of nucleic acid sequences in a random fashion. With the use of appropriate software tools for the assembly of sequence information (e.g., Millikin S. C. et al., Genome Res. 2003, 13: 81-90; Kent, W. J. et al., Genome Res. 2001, 11: 1541-8) it is possible to make use of the knowledge that the “paired-end,” “pairwise,” “paired tag” or “mate pair” sequences are not completely random, but are known or anticipated to occur some distance apart and/or to have some other relationship, and are therefore linked or paired with respect to their position within the genome. This information can aid in the assembly of whole nucleic acid sequences into a consensus sequence.

### Computer-Implemented System

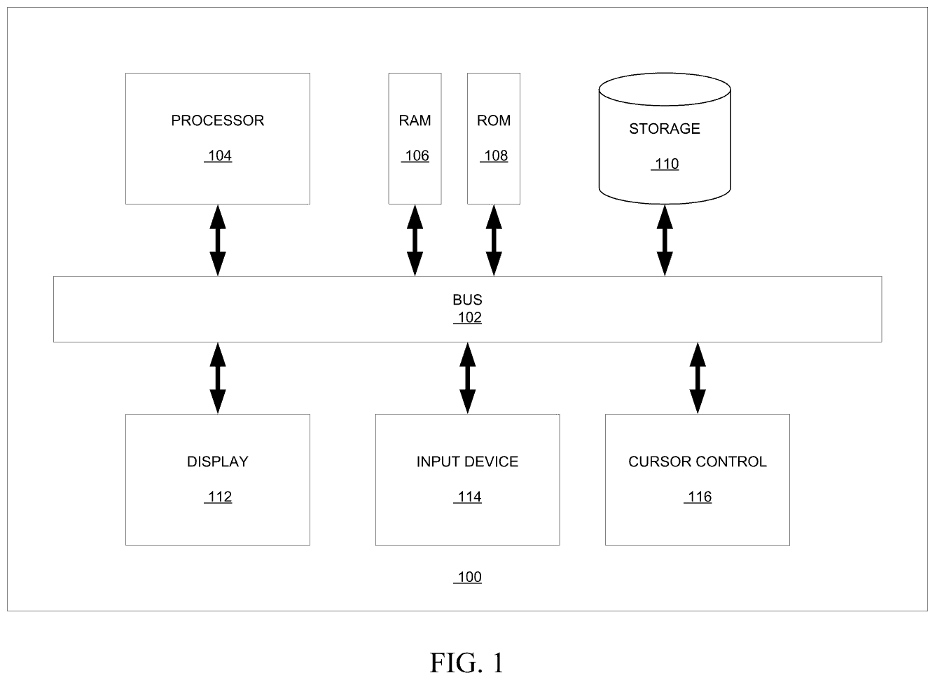

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

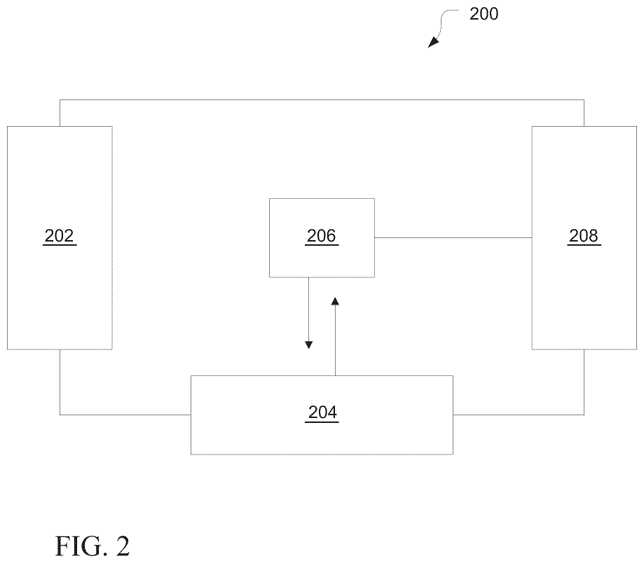

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 (ASN 12/002291), U.S. Patent Application Publication No. 2009/0026082 (ASN 12/002781), U.S. Patent Application Publication No. 2007/066931 (ASN 11/737308) and U.S. Patent Application Publication No. 2008/003571 (ASN 11/345979), which applications are incorporated herein by reference. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques. Ligation sequencing can include single ligation techniques, or change ligation techniques where multiple ligations are performed in sequence on a single primary nucleic acid sequence strand. Sequencing by synthesis can include the incorporation of dye labeled nucleotides, chain termination, ion/proton sequencing, pyrophosphate sequencing, or the like. Single molecule techniques can include continuous sequencing, where the identity of the nuclear type is determined during incorporation without the need to pause or delay the sequencing reaction, or staggered sequence, where the sequencing reactions is paused to determine the identity of the incorporated nucleotide.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

### System and Methods for Identifying Sequence Variation

Identification of sequence variants including single nucleotide polymorphism (SNPs), insertions, and deletions is an important application of next generation sequencing technologies. In various embodiments, the approach/technology implemented during sequencing can influence the accuracy of variant identification. Likewise, the analytical approach used during sequence resolution and alignment can affect the overall quality of the data. For example, in certain embodiments, base space alignment methodologies can misplace or miscall insertions or deletions in the alignment of flow space reads generated using sequencing by synthesis platforms such as the Ion Torrent PGM.

### Example 1: (an Example that May Occur with Increased Frequency)

AAATTT ← reference
AATTTT ← read1
AAATTT ← read2
AATTTT ← read3
AAATTT ← read4

### Example 2: (Another Miss-Aligned Example)

AAACTTT ← reference
AAC--TT ← read5
AAACTTT ← read6
AAC--TT ← read7
AAACTTT ← read8

In the examples above the more likely alignment (explanation) of alignment for reads 1 and 3, showing a deletion of A and an insertion of T, may be as follows:

AAA-TTT ← reference
AA-TTTT ← read1
AA-TTTT ← read3

In various embodiments, although the alignment above may be more likely to be true, it is not necessarily always the correct one. For example, an A→T SNP at the middle position as indicated may not be as rare as expected. Using base space alignment and pileup to select the above alignments, overlooking or misidentifying such types of alignments may occur. In various instances such as the two alignments shown above two forms (mismatch vs undercall+overcall) may be statistically in the same order of magnitude. In such instances, it may be difficult or impractical for an automated sequence or fragment alignment routine to select or identify the most accurate or true candidate. For example, the likelihood of a mismatch occurring may be approximately and the chance of undercall followed by overcall might be large.

From the foregoing discussion it will be appreciated that during sequence analysis a portion of the alignment may be of lower quality. It is therefore not uncommon that the selected analysis path may lead to an incorrect or lower quality result.

In many cases, however, irrespective of the sequence alignment algorithm used for poorly aligned base reads, it may be expected that the bases are not far from their correct positioning/alignments. This can be observed for single bases as well as multiple bases in an exemplary read.

One improved method for basecalling may include applying a Bayesian SNP calling approach configured with a windowing functionality. In various embodiments, such an approach provides a useful mechanism by which to conduct sequence analysis including variant identification such as SNP calling. The Bayesian approach utilizes prior probabilities and the current data to estimate a probability that the read is accurate and not the result of a sequencing error. In various embodiments, the prior probabilities may be determined, at least in part, based on the error modes of the particular sequencing technology used. The Bayesian approach to sequence analysis may therefore be conducted on base space type sequence data as well as on color space data such as that obtained from the SOLiD system and flow space data such as that obtained from the PGM system. One desirable benefit from application of such an approach is that it does not rely on the various bases alignments to be completely and/or necessarily correct.

According to the discussion below addressing an exemplary bi-allelic genome it can be shown that such an approach may be applicable to low frequency sequence variant analysis. Focusing for example on the middle base of example 1 (underlined), a window can be defined and expanded to cover multiple bases, for example, 5 bases surrounding the identified base of interest (the reference in the example window could therefore be assignable to AAATT). For the 4 reads aligned, the sequence bases in the 5 bases projection of the reference can be obtained resulting in sequences of AATTT, AAATT, AATTT, AAATT. In certain embodiments of genomic analysis, the hypothesis for the middle base may be AA, AT with a probability estimate P(O|AA) and P(O|AT), operating under a possible assumption that there is no SNP in this window other than the middle one. This may be reflected by the probability estimate P(AAATT,AATTT,AAATT,AATTT|AAATT) and P(AAATT,AATTT,AAATT,AATTT|AA[A|T]TT). In the former calculation, the assumption may be that there is no SNP at the position, wherein reads 1 and 3 can be explained solely by 2 sequencing errors. In the second term, the assumption may be that there is a SNP [A|T], where a 50%-50% chance suggests that each read is from A or T in the middle, for read 1 and 3, there is 50% chance the read is sequenced correctly and 50% chance it is sequenced with 2 errors, etc. A consideration of the possible situations results in a SNP prior being multiplied to the second term and the hypothesis with the higher probability (thus higher likelihood) will be chosen to represent the genome type at the position.

Under this approach, the SNP caller need not be concerned with which of the two alignments are provided. In such instances, the result will be similar or the same bases and may be reflected by actual error modeling of flowspace data.

Similarly, for example 2, even where a “wrong” alignment is provided, the window approach will consider possible explanations (hypothesis) and determine the likely case where there is no occurrence of the SNP, and there are 2 undercalls in the read.

In the window, the calculation of the probability above may be considered as a local realignment as well. However, in various embodiments each genome type (variant) may be assumed and compared to the local bases. The genome type with the highest match to the local bases can be identified as the most likely sequence for the read

Using an instrument capable of generating flowspace type sequence data such as the PGM, an application may be configured to call indels with a score based on a flow space realignment performed on base space alignment reads. In various embodiments, the flowspace representation may allow for better determination of variants by having different signature between simple intensity differences.

Flow Space Alignment
T, A, C, G, T, A, C, G, T, C, T, G, A, G, C, A, T, C, G, A, T, C, G, A, T, G,
1, 2 , 3, 0, 1 , 2, 3, 0, 1, 1, 0, 0, 2, 0, 3, 0, 1, 0, 0, 1, 0, 4, 0, 0, 1, 0,
|, |, |, |, |, |, |, |, |, , |, |, |, |, |, |, |, |, |, , |, , |, |, |, |,
1, 2 , 3, 0 , 1 , 2 , 3, 0, 1, 0, 0, 0, 2 , 0, 3, 0, 1, 0, 0, 2, 0, 3, 0, 0, 1, 0,

Such differences may be part of the systematic error profile of the sequencing system or reflect actual indel variants. In a more detailed example, a sequence deviation in a read that has only a single marginal flow intensity change supporting a variant may be considered as weak evidence, while one that would require some change in the flow order or multiple strong intensity changes, may be stronger evidence of a variant.

In various embodiments, analysis software may be designed and configured to detect or register multi- or non-positional indel events, by representing deviations as sequences of detected differences between the read query and the target reference in a flow space alignment. From this alignment, calculations of sequence deviations, on a read by read basis may be performed, the deviations found in each read, standardizing the representation of the deviations by merging adjacent deviations together and representing them by a position (for example leftmost).

The analysis approach may then group the variants of multiple reads together by position and produces a score based on flow space alignment characteristics. Alignment characteristics for the score may include intensity differences, missing flow bases, and added flow bases, some or all of which may be compared with what would be expected from a reference target sequence. In certain cases, an additional characteristic may be considered relating to how close the flow(s) that specify the sequence deviation are to either the start or end flow for the read. Such a strategy may help make the score representative of the likelihood of the indel event directly without the need for heuristic filtering. Heuristic knowledge may be considered important, however, and that knowledge, as it is amassed, may provide motivation for greater refinement of the scoring method.

Finally, for heterozygous and rare variant detection, the analytical approach may be configured to determine a selected representation (for example rightmost), and, with the reads fully aligned, determine the number of reads that span the variant (for example, indels).

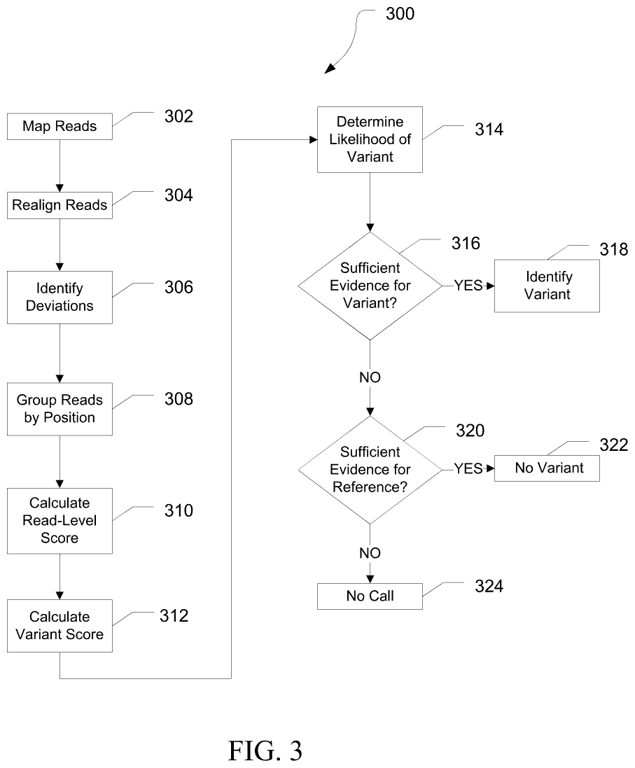

FIG. 3 is an exemplary flow diagram showing a method 300 for identifying variants in nucleic acid sequence reads, in accordance with various embodiments. Using a flow based representation to combine simple intensity differences and flow order differences into a single model, which can result in improved variant calling. In more detail, a sequence deviation in a read that had only a single marginal flow intensity change supporting a variant can provide weak evidence of a variant, whereas a deviation that required a change in flow order or multiple strong intensity changes can provide strong evidence of a variant.

At 302, reads can be mapped to a reference genome. Various algorithms are known in the art for mapping reads to a reference genome. In particular embodiments, the mapping to the reference genome can be performed in base space after the reads are converted from flow space to base space.

At 304, the mapped reads can be realigned to the reference sequence in flow space. In particular embodiments, the portion of the reference sequence to which a read is mapped can be converted into flow space based upon the flow order and the sequence. The flow space representation of the reference can be aligned to the flow space for the read. At 306, deviations in the aligned flow space can be identified on a read-by-read basis. A standardized representation of the deviations can be generated by merging adjacent deviations and representing them in the leftmost position. At 308, variants of multiple reads can be grouped together.

At 310, a read score can be calculated on per-read and per-variant basis. The per-read, per-variant score can be based on flow-space alignment characteristics, such as intensity differences, missing flow bases, and added flow bases as compared to the flow space representation of the reference. Additionally, the per-read, per-variant score can be further based on the location of the variant within the read, such as a distance from the start of the read or a distance from the end of a read. At 312, a variant score can be calculated on a per-variant basis based on the read scores of the reads that span the variant position.

At 314, the likelihood of a variant can be determined. Statistical modeling can be used to determine the probability of a variant for positions containing evidence of a variant. At 316, it can be determined if there is sufficient evidence for the variant at a position, such as a p-value for the variant being below a predefined threshold. When there is sufficient evidence for the variant at the position, the variant can be identified as shown at 318. Alternatively, at 320, when there is not sufficient evidence for a variant at the position, it can be determined if there is sufficient evidence for the read matching the reference sequence at the position. When there is sufficient evidence for the read matching the reference at the position, the position can be identified as having no variant, as shown at 322. Alternatively, at 324, when there is not sufficient evidence for the read matching the reference at the position, a no call can be made for the position, indicating there is not sufficient evidence to identify the position as either the reference or a variant.

In various embodiments, two lists of variants can be generated. The first list can be a list of confident variants, such as those identified at 318. The second list can be candidate variants, such as those positions in which there is insufficient evidence for either a variant call or a reference call. In particular embodiments, a second set of statistical cutoffs can be used to refine the candidate variant list to include positions in which there may not be enough evidence to confidently call either a variant or reference but where there is more evidence for the variant.

In various embodiments, rather than identifying each of the deviations, a list of alleles can be used. The list of alleles can define a position and a variation which can be scored according to the method starting at 310. For each allele in the list of alleles, a call can be made as to whether the allele is present, the position matches the reference, or there in insufficient evidence to call the position. In particular embodiments, the list of alleles can be a list of known alleles that have been previously identified. For example, the known alleles may have been previously identified as relevant to a particular disease or set of diseases. When using a list of alleles, the statistical cutoffs may be changed, for example based on a prior probability that the allele is known to exist in a population.

In various embodiments, a read score can be calculated according to Equation 1 where nf_{deletion} is the number of flows added to represent the reference, nfᵢₙₛₑᵣₜᵢₒₙ is the number of non-empty flows that have no reference, df_{lows} is the sum of the square distances between the read and reference as defined by Equation 2, and coef_{deletion}, coefᵢₙₛₑᵣₜᵢₒₙ, and coef_{intensity} are predefined coefficients. By way of an example, the coef_{deletion} can be in a range of about 1 to about 100, such as for example about 10, coefᵢₙₛₑᵣₜᵢₒₙ can be in a range of about 1 to about 100, such as for example about 10, and coef_{intensity} can be in a range of about 0 to about 10, such as for example about 0.1. Further, a variant score can be calculated according to Equation 3 using the per-read, per-variant score for each read spanning the variant.

$$Equation 1$$

$${Score}_{seq . dev} = 1 + ( {coef}_{deletion} \times {nf}_{deletion} ) + \frac{{coef}_{insertion} \times {nf}_{insertion}}{4} + ( {coef}_{intensity} \times {d}_{flows} )$$

$${d}_{flows} = \sum {( {inten}_{read} - {inten}_{ref} )}^{2} \qquad Equation 2$$

$${Score}_{Variant} = average ( {Scores}_{seq . dev} ) \times log ( \vert Scores \vert ) \qquad Equation 3$$

In various embodiments, a read score can be determined by calculating a Bayesian posterior probability score can be calculated on a per-read, per-variant bases. For each read, P(r|H0) and P(r|H1) can be calculated where H0 is the null hypothesis that there is no variant at a position, and H1 is the predicted variant. The calculation can model the sequence error since the true reference for the read is known. Additionally, the calculation can utilize the reference context surrounding the position and the neighboring flow signals. Log(P(r|H0))−log(P(r|H1)) can estimate the log likelihood that read r is actually sequenced from H1. An average error rate can be determined by taking the sum of the log likelihood of the reads spanning the position. The expected number of reads that may support the hypothesis H1 can be calculated from the average error rate. From the actual number of reads supporting hypothesis H1, the likelihood of the variant at the position can be estimated based on a Poisson distribution.

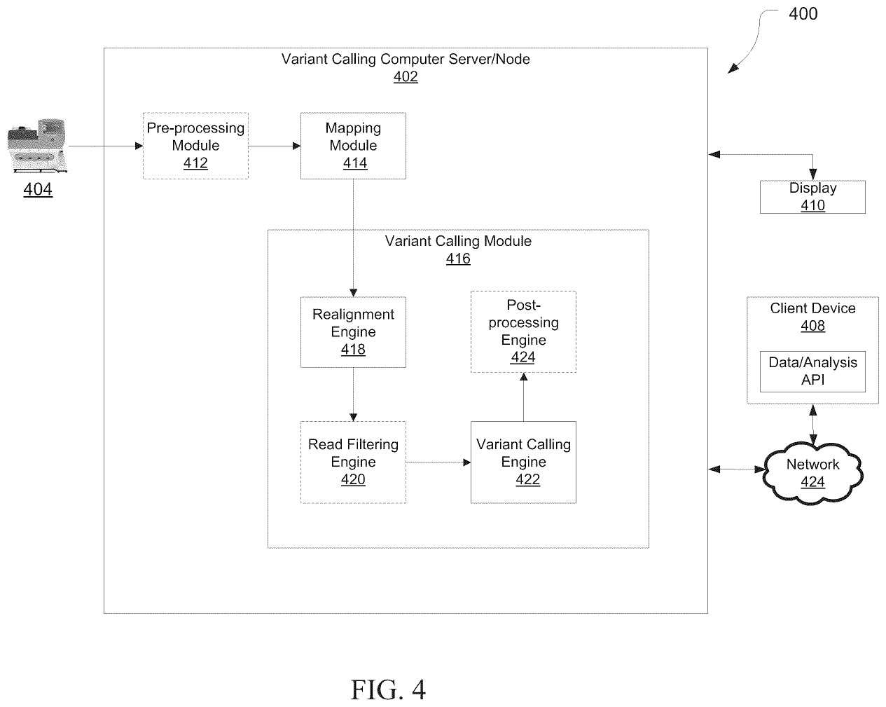

FIG. 4 is a schematic diagram of a system for identifying variants, in accordance with various embodiments.

As depicted herein, variant analysis system 400 can include a nucleic acid sequence analysis device 404 (e.g., nucleic acid sequencer, real-time/digital/quantitative PCR instrument, microarray scanner, etc.), an analytics computing server/node/device 402, and a display 410 and/or a client device terminal 408.

In various embodiments, the analytics computing sever/node/device 402 can be communicatively connected to the nucleic acid sequence analysis device 404, and client device terminal 408 via a network connection 424 that can be either a “hardwired” physical network connection (e.g., Internet, LAN, WAN, VPN, etc.) or a wireless network connection (e.g., Wi-Fi, WLAN, etc.).

In various embodiments, the analytics computing device/server/node 402 can be a workstation, mainframe computer, distributed computing node (part of a “cloud computing” or distributed networking system), personal computer, mobile device, etc. In various embodiments, the nucleic acid sequence analysis device 404 can be a nucleic acid sequencer, real-time/digital/quantitative PCR instrument, microarray scanner, etc. It should be understood, however, that the nucleic acid sequence analysis device 404 can essentially be any type of instrument that can generate nucleic acid sequence data from samples obtained from an individual.

The analytics computing server/node/device 402 can be configured to host an optional pre-processing module 412, a mapping module 414, and a variant calling module 416.

Pre-processing module 412 can be configured to receive from the nucleic acid sequence analysis device 404 and perform processing steps, such as conversion from f space to base space or from flow space to base space, determining call quality values, preparing the read data for use by the mapping module 414, and the like.

The mapping module 414 can be configured to align (i.e., map) a nucleic acid sequence read to a reference sequence. Generally, the length of the sequence read is substantially less than the length of the reference sequence. In reference sequence mapping/alignment, sequence reads are assembled against an existing backbone sequence (e.g., reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, etc.

In various embodiments, the sequence read and reference sequence can be represented as a sequence of nucleotide base symbols in base space. In various embodiments, the sequence read and reference sequence can be represented as one or more colors in color space. In various embodiments, the sequence read and reference sequence can be represented as nucleotide base symbols with signal or numerical quantitation components in flow space.

In various embodiments, the alignment of the sequence fragment and reference sequence can include a limited number of mismatches between the bases that comprise the sequence fragment and the bases that comprise the reference sequence. Generally, the sequence fragment can be aligned to a portion of the reference sequence in order to minimize the number of mismatches between the sequence fragment and the reference sequence.

The variant calling module 416 can include a realignment engine 418, an optional read filtering engine 420, a variant calling engine 422, and an optional post processing engine 424. In various embodiments, variant calling module 416 can be in communications with the mapping module 414. That is, the variant calling module 416 can request and receive data and information (through, e.g., data streams, data files, text files, etc.) from mapping module 414. In various embodiments, the variant calling module 416 can be configured to communicate variants called for a sample genome as a *.vcf, *.gff, or *.hdf data file. It should be understood, however, that the called variants can be communicated using any file format as long as the called variant information can be parsed and/or extracted for later processing/analysis.

The realignment engine 418 can be configured to receive mapped reads from the mapping module 414, realign the mapped reads in altspace, and provide the altspace alignments to the read filtering engine 420.

The read filtering engine 420 can be configured to receive mapped reads from the mapping module 414, filter the reads, calls, and positions based on various criteria, and provide the filtered mapped reads to the variant calling engine 422. Examples of the criteria used to filter the reads, calls, and positions can include mapping quality values, call quality values, a ratio of filtered reads to raw reads, quality values for a non-reference allele, a frequency of a less common allele, coverage of a position, a number of unique start positions for reads that map to a position, presence of an allele in reads from both strands, a number of unique start positions for reads containing the less common allele, the average call quality value for the less common allele, the difference between the average call quality value for the less common allele and the average call quality value for the most common allele, and combinations thereof.

The variant calling engine 422 can be configured to receive filtered altspace alignments from the read filtering engine 420 and analyze the altspace alignments to detect and call (i.e., identify) one or more genomic variants within the reads. Examples of genomic variants that can be called by a variant calling engine 422 include but are not limited to: single nucleotide polymorphisms (SNP), nucleotide insertions or deletions (indels), copy number variations (CNV) identification, inversion polymorphims, etc.

Post processing engine 424 can be configured to receive the variants identified by the variant calling engine 422 and perform additional processing steps, such as conversion from flow space to base space, filtering adjacent variants, and formatting the variant data for display on display 410 or use by client device 408. Examples of filters that the post-processing engine 424 may apply include a minimum score threshold, a minimum number of reads including the variant, a minimum frequency of reads including the variant, a minimum mapping quality, a strand probability, and region filtering.

Client device 408 can be a thin client or thick client computing device. In various embodiments, client terminal 408 can have a web browser (e.g., INTERNET EXPLORER™, FIREFOX™, SAFARI™, etc) that can be used to communicate information to and/or control the operation of the pre-processing module 412, mapping module 414, realignment engine 418, read filtering engine 420, variant calling engine 422, and post processing engine 424 using a browser to control their function. For example, the client terminal 408 can be used to configure the operating parameters (e.g., match scoring parameters, annotations parameters, filtering parameters, data security and retention parameters, etc.) of the various modules, depending on the requirements of the particular application. Similarly, client terminal 408 can also be configure to display the results of the analysis performed by the variant calling module 416 and the nucleic acid sequencer 404.

It should be understood that the various data stores disclosed as part of system 400 can represent hardware-based storage devices (e.g., hard drive, flash memory, RAM, ROM, network attached storage, etc.) or instantiations of a database stored on a standalone or networked computing device(s).

It should also be appreciated that the various data stores and modules/engines shown as being part of the system 400 can be combined or collapsed into a single module/engine/data store, depending on the requirements of the particular application or system architecture. Moreover, in various embodiments, the system 400 can comprise additional modules, engines, components or data stores as needed by the particular application or system architecture.

In various embodiments, the system 400 can be configured to process the nucleic acid reads in color space. In various embodiments, system 400 can be configured to process the nucleic acid reads in base space. In various embodiments, system 400 can be configured to process the nucleic acid sequence reads in flow space. It should be understood, however, that the system 400 disclosed herein can process or analyze nucleic acid sequence data in any schema or format as long as the schema or format can convey the base identity and position of the nucleic acid sequence.

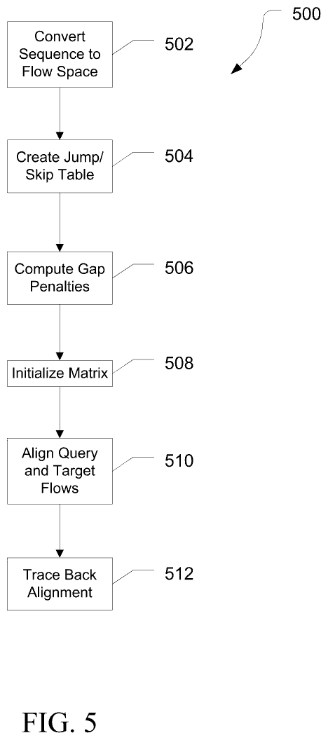

FIG. 5 is an exemplary flow diagram showing a method 500 for aligning reads in flow space.

At 502, a target base sequence can be converted into flow space. The target base sequence can be a portion of the reference sequence to which a read has been mapped. For example, a flow signal vector and a target flow order can be generated from the target base sequence. The target flow order can represent the identities of each base of the target base sequence in order collapsing the repeating bases into a single identity with the flow signal vector can represent the number of times a base is repeated. For example, the target sequence “ACGGATAGG” can create a flow signal vector of “1,1,2,1,1,1,2,1,1,1” with flow order “A, C, G, A, T, A, G”.

At 504, a jump/skip table can be created for the query flow order. The query flow order can be the flow order used by the sequencing instrument when generating the reads. The jump/skip table can represent the number of bases to reach the next index within the query flow order with the same base. Similarly, a reverse jump/skip table can be calculated for the reverse direction. For example, the flow order “T, A, C, G, T, G, C, A” could have the following jump tables:

Jump/Skip Table

| Flow Order | T | A | C | G | T | G | C | A |
|---|---|---|---|---|---|---|---|---|
| Forward | +4 | +6 | +4 | +2 | +4 | +6 | +4 | +2 |
| Reverse | −4 | −2 | −4 | −6 | −4 | −2 | −4 | −6 |

At 506, gap penalties can be pre-computed. The gap penalty can be based on the reverse jump/skip table. For example, the flow order “T, A, C, G, T, G, C, A” and flow signal vector “1,0,1,0,1,0,0,1,1,0,1,0,1,0,0,1” could have the following gap penalties:

Gap Penalties Table

| Flow Order | T | A | C | G | T | G | C | A | T | A | C | G | T | G | C | A |
|---|---|---|---|---|---|---|---|---|---|---|---|---|---|---|---|---|
| Forward | 1 | 0 | 1 | 0 | 1 | 0 | 0 | 1 | 1 | 0 | 1 | 0 | 1 | 0 | 0 | 1 |
| Reverse | 1 | 1 | 2 | 2 | 3 | 1 | 2 | 3 | 3 | 2 | 3 | 3 | 3 | 1 | 2 | 3 |

At 508, the dynamic programming matrix can be initialized. The dynamic programming matrix can be initialized such that a cell within the matrix stores a match score and match traceback, an insertion score and insertion traceback, a deletion score and deletion traceback. The start cells can be initialized with the phase penalty and pre-computed gap penalties.

At 510, the read flow information can be aligned with the target flow information. A dynamic programming algorithm can loop over the flows in the query flow signal vector and the target flow signal vector. As the dynamic programming algorithm progresses, the possible moves can be considered, a horizontal move, a vertical move, and a diagonal move. The horizontal move corresponds to skipping over a target flow base which would represent a deletion in the read. The information from the previous column and same row can be extended in to the current cell and can be weighted by the target flow signal that was skipped. For the vertical move, when the query flow base and the target flow base do not match, the target flow order can be padded with empty flows having a flow signal of 0. Additionally, the previous rows score can be penalized by the query flow signal for the current base. Additionally, the previous matching base in the query flow order can be considered. A phase penalty and a pre-computed gap penalty can be added, the move with the maximum score can be kept and the traceback cell can be annotated appropriately. For the diagonal move can be considered when the query flow base and the target flow base match. The score can be determined from the absolute value of the difference between the query flow signal and the target flow signal.

At 512, the alignment can be determined by tracing back along the highest scoring path. When the path includes a match, the flow signals from the query and target can be pushed into the alignment. When the path includes an insertion, for an empty target flow the query flow signal, the empty target signal, and the query flow base can be added to the alignment. Alternatively, for a phased insertion, the query flow bases, the query flow signals, and the target gaps back to the previous matching query flow base can be added to the alignment. When the path includes a deletion, a query gap, the target flow signal, and the target flow base can be added to the alignment. Once the alignment has been traced back, the alignment can be reversed to obtain the alignment in the forward direction

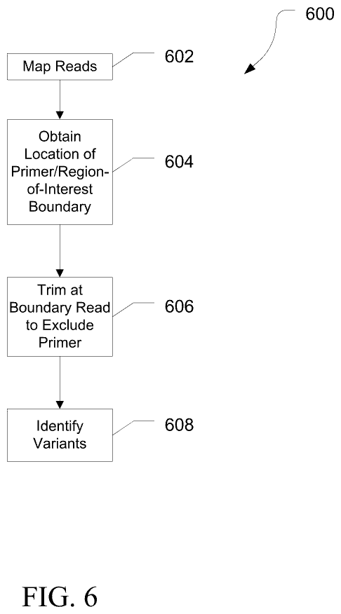

FIG. 6 is an exemplary flow diagram showing a method 600 for trimming primer sequences from reads. The sequence within the primer region may not match the genetic sequence of the individual due to mismatches between the primer and the genetic sequence, which could lead to falsely identifying variants.

At 602, the reads can be mapped to a reference genome.

At 604, the location of the boundary between the primer and the region of interest can be obtained. For example, a file can be provided with a listing of the location of the primers within the reference genome and the boundary locations can be determined based on the ends of the primers.

Alternatively, the boundary region can be identified from the alignment of forward and reverse reads of an amplicon. The primer region of a read may not match the primer sequence as, for at least some reads, it may correspond to the reference sequence. However, by aligning the forward and reverse reads for an amplicon, the downstream primer region for a forward read aligns with the upstream primer region of the reverse read.

At 606, the reads can be trimmed at the boundary locations to exclude the primers. In particular embodiments, the primers can be marked so that the sequence information is retained but excluded from being used in variant identification.

At 608, variants within the regions of interest can be identified.

Various embodiments, disclosed herein, comprise Bayesian and frequentist algorithms with data filters tuned to sensitive and specific detection of low frequency variants in a sample. In various embodiments, the low frequency variant detection systems and methods disclosed herein can be utilized in a variety of applications, including but not limited to the detection of somatic mutations in: tumor samples, pooled samples, metagenomics, novel mutations, fetal DNA in a background of maternal DNA, mitochondrial heteroplasmy and heterogeneous samples, etc.

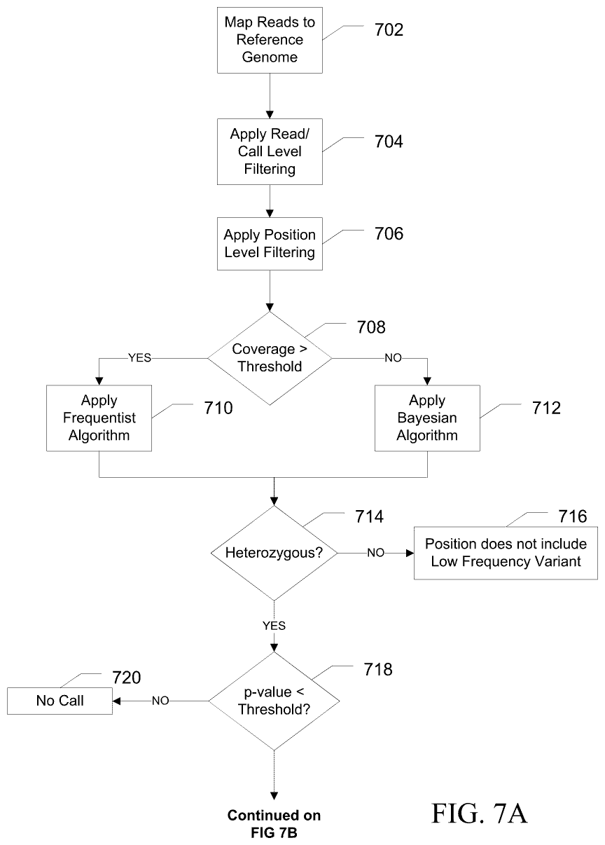

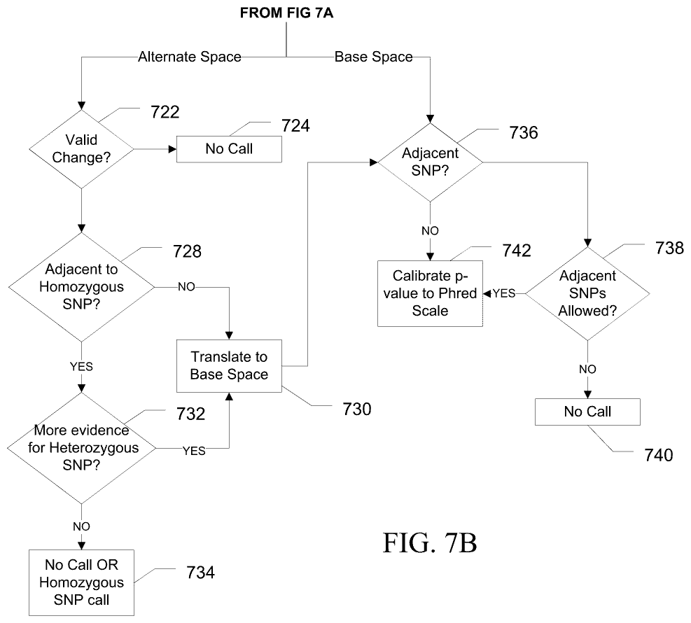

FIGS. 7A and 7B are exemplary flowcharts showing a method 700 for detecting low frequency variants in nucleic acid sequence reads, in accordance with various embodiments. When there are at least two different calls for a position, low frequency variant analysis can determine if a less common call is likely due to a read or sequencing error or a heterozygous sample. At 702, reads can be mapped to a reference genome. Various algorithms are known in the art for mapping reads to a reference genome. At 704, the reads and positions can be filtered based on a quality of a call, such as a color call or a base call, or a quality of a mapping. For example, when a mapping quality value is below a threshold, the mapped read can be excluded from further analysis. In a further example, when a call quality value (a color quality value or a base quality value) is below a threshold, the read can be included in further variant analysis, but the base call or color call can be excluded.

At 706, position level filtering can be applied to the mapped locations. A general filter can be applied to determine if a position should be considered for further variant analysis. The general filter can exclude positions where the ratio of the filtered reads to raw reads is below a threshold, thereby excluding positions where are large fraction of the reads that are mapped to the position have been discarded based on poor mapping quality. Further, the quality values for each call that is mapped to a position but does not match the reference base or color can be averaged and the general filter can exclude positions where the average call quality value for non-reference calls is below a threshold.

Additionally, a set of low frequency variant filters can be applied to determine if a position should be considered for low frequency variant analysis. The low frequency variant filter can exclude a position when the coverage of the position is below a coverage threshold. Further, a position can be excluded when the number of unique start positions for reads that map to the position is below a pile-up threshold.

The low frequency variant filter can exclude a less common allele (alternate call) when the percentage of reads of the less common allele is below an allele frequency threshold. Less common alleles (LCA) can also be excluded when the less common allele is not present on both strands, when the number of unique starting positions is below a LCA pile-up threshold, when the average call quality for the less common allele is below a LCA QV threshold, when the maximum difference between the average call quality for the less common allele and the average call quality for the most common allele exceeds a QV difference threshold, or any combination thereof.

At 708, when an allele at a position is not excluded by the above filters, a determination can be made if the coverage is above a threshold. When the coverage is above the threshold, a frequentist algorithm can be applied to determine a probability that the variant is not a read error, as illustrated at 710. Alternatively, when the coverage is below the threshold, a Bayesian algorithm can be applied to determine a probability that the variant is not a read or sequencing error, as illustrated at 712.

At 714, it can be determined if the position is heterozygous (more than one allele for the position is in the sample). When the position is not heterozygous, such as when a position has only one allele that is not excluded by the filters, the position does not include a low frequency variant, as illustrated at 716. Alternatively, at 718, when more than one allele is identified for a position, the probability calculated at either 710 or 712 can be compared to a probability threshold. In various embodiments, at 720, when the p-value is less than the probability threshold, no call may be made for the position. Alternatively, when the p-value is not less than the threshold, the method can proceed to FIG. 7B.

Turning to FIG. 7B, at 722, when the reads are in altspace, a check can be made to determine if the read is a valid altspace read. For example, when using a two-base color code, certain color sequences, such a single position color change, may not be valid for a read. Similarly, in flow space, two consecutive incorporation events of the same base may not be valid for a read. When a variant not valid in altspace, a call may not be made for the variant, as illustrated at 724. Alternatively, when the variant is valid in altspace, positions with a heterozygous variant can be checked to determine if they are adjacent to a homozygous variant, as illustrated at 726. When a heterozygous position is not adjacent to a homozygous position, at 728, the read can be converted to base space, as illustrated at 730. When a heterozygous position is adjacent to a homozygous position, at 732, the evidence for the heterozygous position can be compared to the evidence for the homozygous position. When the evidence for the heterozygous position does not have more evidence than the homozygous position, a homozygous variant call can be made or no variant call may be made, as illustrated at 734. Alternatively, at 730, when there is more evidence for the heterozygous position than for the homozygous position, the read can be converted into base space.

At 736, when the reads are in base space or after the variant call is translated into base space, a variant can be checked to determine if it is adjacent to another variant. When the position with a variant is adjacent to another position with a variant, at 738, user settings can be checked to determine if adjacent variants are allowed. When adjacent variants are not allowed, no variant call may be made at the position, as illustrated at 740. At 742, when the position with the variant is not adjacent to another position or when adjacent variants are allowed, the p-value can be calibrated to the Phred scale, and the low frequency variant can be reported with a variant quality value.

In various embodiments, the methods of the present teachings may be implemented in a software program and applications written in conventional programming languages such as C, C++, etc.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

1. A system to identify variants in nucleic acid sequence data provided by a nucleic acid sequence analysis device, comprising:
a processor in communication with the nucleic acid sequence analysis device, the processor configured to execute machine-readable instructions, said instructions which when executed cause the processor to perform the following steps:
map a plurality of nucleic acid sequence reads to a reference genome to form mapped reads,
align flow space information for the mapped reads to a flow space representation of at least a portion of the reference genome to form aligned flow space information,
identify sequence deviations between the aligned flow space information and the flow space representation of the reference genome,
group the sequence deviations identified for multiple reads by position,
calculate a read score for each of the sequence deviations for a position on a per-read and per-variant basis using characteristics of the aligned flow space information and based on a location of the sequence deviation within the read,
calculate a variant score for the sequence deviations at the position on a per-variant basis based on the read scores spanning the position, and
identify a variant at the position if there is sufficient evidence of the variant at the position.

2. The system of claim 1, wherein the step to calculate the variant score includes calculating an average of the read scores.

3. The system of claim 1, wherein the step to calculate the variant score includes calculating a Bayesian posterior probability on a per-read and per variant basis.

4. The system of claim 1, wherein the instructions further include a step to indicate whether the mapped read matches the reference genome at the position.

5. The system of claim 4, wherein the step to indicate includes identifying a no-variant status at the position when there is sufficient evidence of a match between the mapped read and the reference genome.

6. The system of claim 4, wherein the step to indicate includes identifying a no-call status at the position when there is not sufficient evidence of a match between the mapped read and the reference genome.

7. The system of claim 1, wherein the step to map a plurality of nucleic acid sequence reads to a reference genome uses base space representations of the plurality of nucleic acid sequence reads and a base space representation of the reference genome to form the mapped reads.

8. The system of claim 7, wherein the step to align flow space information for the mapped reads to a flow space representation of at least a portion of the reference genome includes converting a portion of the base space representation of the reference genome corresponding to the mapped reads to the flow space representation of the reference genome.

9. The system of claim 8, wherein the converting uses a flow order and an order of bases in the base space representation of the reference genome to determine the flow space representation of the reference genome.

10. A computer implemented method for identifying variants in nucleic acid sequence data provided by a nucleic acid sequence analysis device, comprising:
receiving, at a processor in communication with the nucleic acid sequence analysis device, a plurality of nucleic acid sequence reads;
mapping the plurality of nucleic acid sequence reads to a reference genome to form mapped reads;
aligning flow space information for the mapped reads to a flow space representation of at least a portion of the reference genome to form aligned flow space information;
identifying sequence deviations between the aligned flow space information and the flow space representation of the reference genome;
grouping the sequence deviations identified for multiple reads by position;
calculating a read score for each of the sequence deviations for a position on a per-read and per-variant basis using characteristics of the aligned flow space information and based on a location of the sequence deviation within the read;
calculating a variant score for the sequence deviations at the position on a per-variant basis based on the read scores spanning the position; and
identifying a variant at the position if there is sufficient evidence of the variant at the position.

11. The method of claim 10, wherein calculating the variant score includes calculating a Bayesian posterior probability on a per-read and per variant basis.

12. The method of claim 10, wherein the calculating a variant score includes calculating an average of the read scores.

13. The method of claim 10, further comprising indicating whether the mapped read matches the reference genome at the position.

14. The method of claim 13, further comprising identifying a no-variant status at the position when there is sufficient evidence of a match between the mapped read and the reference genome.

15. The method of claim 13, further comprising identifying a no-call status at the position when there is not sufficient evidence of a match between the mapped read and the reference genome.

16. The method of claim 10, wherein the mapping uses base space representations of the plurality of nucleic acid sequence reads and a base space representation of the reference genome to form the mapped reads.

17. The method of claim 16, wherein the aligning flow space information for the mapped reads to a flow space representation of at least a portion of the reference genome includes converting a portion of the base space representation of the reference genome corresponding to the mapped reads to the flow space representation of the reference genome.

18. The method of claim 17, wherein the converting uses a flow order and an order of bases in the base space representation of the reference genome to determine the flow space representation of the reference genome.

19. A non-transitory machine-readable storage medium comprising instructions which, when executed by a processor, cause the processor to perform a method, comprising:
receiving, at the processor, a plurality of nucleic acid sequence reads from a nucleic acid sequence analysis device;
mapping the plurality of nucleic acid sequence reads to a reference genome to form mapped reads;
aligning flow space information for the mapped reads to a flow space representation of at least a portion of the reference genome to form aligned flow space information;
identifying sequence deviations between the aligned flow space information and the flow space representation of the reference genome;
grouping the sequence deviations identified for multiple reads by position;
calculating a read score for each of the sequence deviations for a position on a per-read and per-variant basis using characteristics of the aligned flow space information and based on a location of the sequence deviation within the read;
calculating a variant score for the sequence deviations at the position on a per-variant basis based on the read scores spanning the position; and
identifying a variant at the position if there is sufficient evidence of the variant at the position.

20. The non-transitory machine-readable storage medium of claim 19, wherein calculating the variant score includes calculating a Bayesian posterior probability on a per-read and per variant basis.
